SEQUENCE LISTING (31 sequences)

SEQ ID NO: 1 - protein, 17 aa, Artificial sequence
  Description of Artificial Sequence AviTagTM biotinylation polypeptide, substrate for holoenzyme synthetase (BirA) of E.coli.
MSGLNDIFEAQKIEWHE

SEQ ID NO: 2 - DNA, 51 bp, Artificial sequence
  Description of Artificial Sequence Consensus sequence for optimized nucleic acid sequences encoding the biotinylation polypeptide of SEQ ID NO 1.
atgwsygghytraaygayatyttygaggcwcagaaaatcgaatggcacgaa

SEQ ID NO: 3 - DNA, 51 bp, Artificial sequence
  Description of Artificial Sequence Nucleic acid sequence encoding the biotinylation polypeptide of SEQ ID NO 1; AviTagTM wild-type sequence.
atgtccggcctgaacgacatcttcgaggctcagaaaatcgaatggcacgaa

SEQ ID NO: 4 - DNA, 51 bp, Artificial sequence
  Description of Artificial Sequenceencoding the biotinylation polypeptide of SEQ ID NO 1 for enhanced expression of biotinylated fusion polypeptides.
atgagtggattaaatgatatttttgaagcacagaaaatcgaatggcacgaa

SEQ ID NO: 5 - DNA, 51 bp, Artificial sequence
  Description of Artificial Sequenceencoding the biotinylation polypeptide of SEQ ID NO 1 for enhanced expression of biotinylated fusion polypeptides.
atgagtggattaaatgatattttcgaagcacagaaaatcgaatggcacgaa

SEQ ID NO: 6 - DNA, 51 bp, Artificial sequence
  Description of Artificial Sequenceencoding the biotinylation polypeptide of SEQ ID NO 1 for enhanced expression of biotinylated fusion polypeptides.
atgagtggtttaaatgatattttcgaggctcagaaaatcgaatggcacgaa

SEQ ID NO: 7 - DNA, 51 bp, Artificial sequence
  Description of Artificial Sequenceencoding the biotinylation polypeptide of SEQ ID NO 1 for enhanced expression of biotinylated fusion polypeptides.
atgagtggcttaaatgatattttcgaggctcagaaaatcgaatggcacgaa

SEQ ID NO: 8 - DNA, 51 bp, Artificial sequence
  Description of Artificial Sequenceencoding the biotinylation polypeptide of SEQ ID NO 1 for enhanced expression of biotinylated fusion polypeptides.
atgagtggtttaaatgatatcttcgaggctcagaaaatcgaatggcacgaa

SEQ ID NO: 9 - DNA, 51 bp, Artificial sequence
  Description of Artificial Sequenceencoding the biotinylation polypeptide of SEQ ID NO 1 for enhanced expression of biotinylated fusion polypeptides.
atgagcggtttaaatgatattttcgaggctcagaaaatcgaatggcacgaa

SEQ ID NO: 10 - DNA, 51 bp, Artificial sequence
  Description of Artificial Sequenceencoding the biotinylation polypeptide of SEQ ID NO 1 for enhanced expression of biotinylated fusion polypeptides.
atgagtggtttaaatgacattttcgaggctcagaaaatcgaatggcacgaa

SEQ ID NO: 11 - DNA, 51 bp, Artificial sequence
  Description of Artificial Sequenceencoding the biotinylation polypeptide of SEQ ID NO 1 for enhanced expression of biotinylated fusion polypeptides.
atgagtggcttaaatgatatcttcgaggctcagaaaatcgaatggcacgaa

SEQ ID NO: 12 - DNA, 51 bp, Artificial sequence
  Description of Artificial Sequenceencoding the biotinylation polypeptide of SEQ ID NO 1 for enhanced expression of biotinylated fusion polypeptides.
atgagtggtttaaacgatattttcgaggctcagaaaatcgaatggcacgaa

SEQ ID NO: 13 - DNA, 51 bp, Artificial sequence
  Description of Artificial Sequenceencoding the biotinylation polypeptide of SEQ ID NO 1 for enhanced expression of biotinylated fusion polypeptides.
atgtctggtttaaatgatattttcgaggctcagaaaatcgaatggcacgaa

SEQ ID NO: 14 - DNA, 51 bp, Artificial sequence
  Description of Artificial Sequenceencoding the biotinylation polypeptide of SEQ ID NO 1 for enhanced expression of biotinylated fusion polypeptides.
atgagtggcttaaatgacattttcgaggctcagaaaatcgaatggcacgaa

SEQ ID NO: 15 - DNA, 51 bp, Artificial sequence
  Description of Artificial Sequenceencoding the biotinylation polypeptide of SEQ ID NO 1 for enhanced expression of biotinylated fusion polypeptides.
atgagcggtttaaatgatatcttcgaggctcagaaaatcgaatggcacgaa

SEQ ID NO: 16 - DNA, 51 bp, Artificial sequence
  Description of Artificial Sequenceencoding the biotinylation polypeptide of SEQ ID NO 1 for enhanced expression of biotinylated fusion polypeptides.
atgagcggtttaaacgatattttcgaggctcagaaaatcgaatggcacgaa

SEQ ID NO: 17 - DNA, 51 bp, Artificial sequence
  Description of Artificial Sequenceencoding the biotinylation polypeptide of SEQ ID NO 1 for enhanced expression of biotinylated fusion polypeptides.
atgagcggcttaaatgatattttcgaggctcagaaaatcgaatggcacgaa

SEQ ID NO: 18 - DNA, 51 bp, Artificial sequence
  Description of Artificial Sequenceencoding the biotinylation polypeptide of SEQ ID NO 1 for enhanced expression of biotinylated fusion polypeptides.
atgtctggtttaaatgatatcttcgaggctcagaaaatcgaatggcacgaa

SEQ ID NO: 19 - DNA, 51 bp, Artificial sequence
  Description of Artificial Sequenceencoding the biotinylation polypeptide of SEQ ID NO 1 for enhanced expression of biotinylated fusion polypeptides.
atgagtggtttaaacgatatcttcgaggctcagaaaatcgaatggcacgaa

SEQ ID NO: 20 - DNA, 51 bp, Artificial sequence
  Description of Artificial Sequenceencoding the biotinylation polypeptide of SEQ ID NO 1 for enhanced expression of biotinylated fusion polypeptides.
atgagcggcttaaatgatatcttcgaggctcagaaaatcgaatggcacgaa

SEQ ID NO: 21 - DNA, 51 bp, Artificial sequence; identical to SEQ ID NO: 20
  Description of Artificial Sequenceencoding the biotinylation polypeptide of SEQ ID NO 1 for enhanced expression of biotinylated fusion polypeptides.

SEQ ID NO: 22 - DNA, 51 bp, Artificial sequence
  Description of Artificial Sequenceencoding the biotinylation polypeptide of SEQ ID NO 1 for enhanced expression of biotinylated fusion polypeptides.
atgagtggcttaaacgatattttcgaggctcagaaaatcgaatggcacgaa

SEQ ID NO: 23 - DNA, 51 bp, Artificial sequence
  Description of Artificial Sequenceencoding the biotinylation polypeptide of SEQ ID NO 1 for enhanced expression of biotinylated fusion polypeptides.
atgagtggtttaaacgacattttcgaggctcagaaaatcgaatggcacgaa

SEQ ID NO: 24 - DNA, 51 bp, Artificial sequence
  Description of Artificial Sequenceencoding the biotinylation polypeptide of SEQ ID NO 1 for enhanced expression of biotinylated fusion polypeptides.
atgagtggcttaaatgacatcttcgaggctcagaaaatcgaatggcacgaa

SEQ ID NO: 25 - DNA, 966 bp, Escherichia coli
  misc_feature: Coding sequence for biotin-holoenzyme synthetase, birA; according to GenBank entry gi145430
atgaaggataacaccgtgccactgaaattgattgccctgttagcgaacggtgaatttcactctggcgagcagttgggtgaaacgctgggaatgagccgggcggctattaataaacacattcagacactgcgtgactggggcgttgatgtctttaccgttccgggtaaaggatacagcctgcctgagcctatccagttacttaatgctaaacagatattgggtcagctggatggcggtagtgtagccgtgctgccagtgattgactccacgaatcagtaccttcttgatcgtatcggagagcttaaatcgggcgatgcttgcattgcagaataccagcaggctggccgtggtcgccggggtcggaaatggttttcgccttttggcgcaaacttatatttgtcgatgttctggcgtctggaacaaggcccggcggcggcgattggtttaagtctggttatcggtatcgtgatggcggaagtattacgcaagctgggtgcagataaagttcgtgttaaatggcctaatgacctctatctgcaggatcgcaagctggcaggcattctggtggagctgactggcaaaactggcgatgcggcgcaaatagtcattggagccgggatcaacatggcaatgcgccgtgttgaagagagtgtcgttaatcaggggtggatcacgctgcaggaagcggggatcaatctcgatcgtaatacgttggcggccatgctaatacgtgaattacgtgctgcgttggaactcttcgaacaagaaggattggcaccttatctgtcgcgctgggaaaagctggataattttattaatcgcccagtgaaacttatcattggtgataaagaaatatttggcatttcacgcggaatagacaaacagggggctttattacttgagcaggatggaataataaaaccctggatgggcggtgaaatatccctgcgtagtgcagaaaaataa

SEQ ID NO: 26 - DNA, 92 bp, Artificial sequence
  Description of Artificial Sequence Forward primer
gtttccctctagaaataattttgtttaactttaagaaggagatataccatgtcnggnytnaaygayathttygargcncagaaaatcgaatg

SEQ ID NO: 27 - DNA, 92 bp, Artificial sequence
  Description of Artificial Sequence Forward primer
gtttccctctagaaataattttgtttaactttaagaaggagatataccatgagyggnytnaaygayathttygargcncagaaaatcgaatg

SEQ ID NO: 28 - DNA, 30 bp, Artificial sequence
  Description of Artificial Sequence Reverse primer
caagtgttggccatggaacaggtagttttc

SEQ ID NO: 29 - DNA, 8572 bp, Artificial sequence
  Description of Artificial Sequence pIVEX-GFP WT AviTagTM; GFP vector with coding sequence for wildtype AviTagTM-GFP fusion polypeptide
tcgcgcgtttcggtgatgacggtgaaaacctctgacacatgcagctcccgagcgcgcaaagccactactgccacttttggagactgtgtacgtcgagggcgagacggtcacagcttgtctgtaagcggatgccgggagcagacaagcccgctctgccagtgtcgaacagacattcgcctacggccctcgtctgttcgggctcagggcgcgtcagcgggtgttggcgggtgtcggggctggcttaactatgagtcccgcgcagtcgcccacaaccgcccacagccccgaccgaattgataccggcatcagagcagattgtactgagagtgcaccatatgcggtgtgaaatagccgtagtctcgtctaacatgactctcacgtggtatacgccacactttatccgcacagatgcgtaaggagaaaataccgcatcaggcgccattcgccattggcgtgtctacgcattcctcttttatggcgtagtccgcggtaagcggtaacaggctgcgcaactgttgggaagggcgatcggtgcgggcctcttcgctatgtccgacgcgttgacaacccttcccgctagccacgcccggagaagcgatatacgccagctggcgaaagggggatgtgctgcaaggcgattaagttgggtaatgcggtcgaccgctttccccctacacgacgttccgctaattcaacccatacgccagggttttcccagtcacgacgttgtaaaacgacggccagtgccaatgcggtcccaaaagggtcagtgctgcaacattttgctgccggtcacggttgcttgcatgcaaggagatggcgcccaacagtcccccggccacggggcctgcgaacgtacgttcctctaccgcgggttgtcagggggccggtgccccggacccaccatacccacgccgaaacaagcgctcatgagcccgaagtggcgagccggtggtatgggtgcggctttgttcgcgagtactcgggcttcaccgctcggcgatcttccccatcggtgatgtcggcgatataggcgccagcaaccgcaccgctagaaggggtagccactacagccgctatatccgcggtcgttggcgtggtgtggcgccggtgatgccggccacgatgcgtccggcgtagaggatcgagaacaccgcggccactacggccggtgctacgcaggccgcatctcctagctcttctcgatcccgcgaaattaatacgactcactatagggagaccacaacggtagagctagggcgctttaattatgctgagtgatatccctctggtgttgccattccctctagaaataattttgtttaactttaagaaggagatataccatgtaagggagatctttattaaaacaaattgaaattcttcctctatatggtacaccggcctgaacgacatcttcgaggctcagaaaatcgaatggcacgaaactggccggacttgctgtagaagctccgagtcttttagcttaccgtgctttgaagcaaaggagaagaacttttcactggagttgtcccaattcttgttgaatttcgtttcctcttcttgaaaagtgacctcaacagggttaagaacaacttaaagatggtgatgttaatgggcacaaattttctgtcagtggagagggtgaagtctaccactacaattacccgtgtttaaaagacagtcacctctcccacttcgtgatgctacatacggaaagcttacccttaaatttatttgcactactggacactacgatgtatgcctttcgaatgggaatttaaataaacgtgatgacctaaactacctgttccatggccaacacttgtcactactttctcttatggtgttttgatggacaaggtaccggttgtgaacagtgatgaaagagaataccacatcaatgcttttcccgttatccggatcatatgaaacggcatgactttttcaagttacgaaaagggcaataggcctagtatactttgccgtactgaaaaagtagagtgccatgcccgaaggttatgtacaggaacgcactatatctttcaaatctcacggtacgggcttccaatacatgtccttgcgtgatatagaaagtttgatgacgggaactacaagacgcgtgctgaagtcaagtttgaaggtgatacctactgcccttgatgttctgcgcacgacttcagttcaaacttccactatgccttgttaatcgtatcgagttaaaaggtattgattttaaagaagatggaaggaacaattagcatagctcaattttccataactaaaatttcttctaccttacattctcggacacaaactcgagtacaactataactcacacaatgtatactgtaagagcctgtgtttgagctcatgttgatattgagtgtgttacatatgatcacggcagacaaacaaaagaatggaatcaaagctaacttcaaaattcgtagtgccgtctgtttgttttcttaccttagtttcgattgaagttttaagcccacaacattgaagatggatccgttcaactagcagaccattatcaacaaaggtgttgtaacttctacctaggcaagttgatcgtctggtaatagttgtttatactccaattggcgatggccctgtccttttaccagacaaccattacctgtatgaggttaaccgctaccgggacaggaaaatggtctgttggtaatggactcgacacaatctgccctttcgaaagatcccaacgaaaagagagaccacatagctgtgttagacgggaaagctttctagggttgcttttctctctggtgtaggtccttcttgagtttgtaacagctgctgggattacacatggcatggatgccaggaagaactcaaacattgtcgacgaccctaatgtgtaccgtacctacaactatacaaacccggggggggttctcatcatcatcatcatcattaataattgatatgtttgggccccccccaagagtagtagtagtagtagtaattattaagggcgaattccagcacactggcggccgttactagtggatccggctgctttcccgcttaaggtcgtgtgaccgccggcaatgatcacctaggccgacgaaacaaagcccgaaaggaagctgagttggctgctgccaccgctgagcaatattgtttcgggctttccttcgactcaaccgacgacggtggcgactcgttatactagcataaccccttggggcctctaaacgggtcttgaggggttttttgctgatcgtattggggaaccccggagatttgcccagaactccccaaaaaacgtgaaaggaggaactatatccggatatccacaggacgggtgtggtcgccatactttcctccttgatataggcctataggtgtcctgcccacaccagcggtagatcgcgtagtcgatagtggctccaagtagcgaagcgagcaggactgggcctagcgcatcagctatcaccgaggttcatcgcttcgctcgtcctgacccgggcggccaaagcggtcggacagtgctccgagaacgggtgcgcatagaaatccgccggtttcgccagcctgtcacgaggctcttgcccacgcgtatctttatgcatcaacgcatatagcgctagcagcacgccatagtgactggcgatgctacgtagttgcgtatatcgcgatcgtcgtgcggtatcactgaccgctacgagtcggaatggacgatatcccgcaagaggcccggcagtaccggcataaccacagccttacctgctatagggcgttctccgggccgtcatggccgtattggtagcctatgcctacagcatccagggtgacggtgccgaggatgacgatgagctcggatacggatgtcgtaggtcccactgccacggctcctactgctactcggcattgttagatttcatacacggtgcctgactgcgttagcaatttaactgcgtaacaatctaaagtatgtgccacggactgacgcaatcgttaaattgactgataaactaccgcattaaagcttatcgatgataagctgtcaaacatgagactatttgatggcgtaatttcgaatagctactattcgacagtttgtactcaattcgtaatcatggtcatagctgtttcctgtgtgaaattgttatccgctttaagcattagtaccagtatcgacaaaggacacactttaacaataggcgacacaattccacacaacatacgagccggaagcataaagtgtaaagcctggggtgttaaggtgtgttgtatgctcggccttcgtatttcacatttcggacccgtgcctaatgagtgagctaactcacattaattgcgttgcgctcactgccccacggattactcactcgattgagtgtaattaacgcaacgcgagtgacggggctttccagtcgggaaacctgtcgtgccagctgcattaatgaatcggccacgaaaggtcagccctttggacagcacggtcgacgtaattacttagccggtacgcgcggggagaggcggtttgcgtattgggcgctcttccgcttcctcgctgcgcgcccctctccgccaaacgcataacccgcgagaaggcgaaggagcgtcactgactcgctgcgctcggtcgttcggctgcggcgagcggtatcagctagtgactgagcgacgcgagccagcaagccgacgccgctcgccatagtcgacactcaaaggcggtaatacggttatccacagaatcaggggataacgcagggtgagtttccgccattatgccaataggtgtcttagtcccctattgcgtccaaagaacatgtgagcaaaaggccagcaaaaggccaggaaccgtaaaaaggtttcttgtacactcgttttccggtcgttttccggtccttggcatttttccccgcgttgctggcgtttttccataggctccgcccccctgacgagcatcacggcgcaacgaccgcaaaaaggtatccgaggcggggggactgctcgtagtgaaaaatcgacgctcaagtcagaggtggcgaaacccgacaggactataaagtttttagctgcgagttcagtctccaccgctttgggctgtcctgatatttcataccaggcgtttccccctggaagctccctcgtgcgctctcctgttccgatatggtccgcaaagggggaccttcgagggagcacgcgagaggacaaggctccctgccgcttaccggatacctgtccgcctttctcccttcgggaagcgtggggacggcgaatggcctatggacaggcggaaagagggaagcccttcgcacgcgctttctcatagctcacgctgtaggtatctcagttcggtgtaggtcgtcgcgaaagagtatcgagtgcgacatccatagagtcaagccacatccagcatcgctccaagctgggctgtgtgcacgaaccccccgttcagcccgaccgctagcgaggttcgacccgacacacgtgcttggggggcaagtcgggctggcgagcgccttatccggtaactatcgtcttgagtccaacccggtaagacacgaccgcggaataggccattgatagcagaactcaggttgggccattctgtgctgttatcgccactggcagcagccactggtaacaggattagcagagcgaggtaaatagcggtgaccgtcgtcggtgaccattgtcctaatcgtctcgctccattgtaggcggtgctacagagttcttgaagtggtggcctaactacggctacaacatccgccacgatgtctcaagaacttcaccaccggattgatgccgatgtctagaaggacagtatttggtatctgcgctctgctgaagccagttaccttcgatcttcctgtcataaaccatagacgcgagacgacttcggtcaatggaagggaaaaagagttggtagctcttgatccggcaaacaaaccaccgctggtagcctttttctcaaccatcgagaactaggccgtttgtttggtggcgaccatccggtggtttttttgtttgcaagcagcagattacgcgcagaaaaaaaggatgccaccaaaaaaacaaacgttcgtcgtctaatgcgcgtctttttttcctactcaagaagatcctttgatcttttctacggggtctgacgctcagtggaacgagttcttctaggaaactagaaaagatgccccagactgcgagtcaccttggaaaactcacgttaagggattttggtcatgagattatcaaaaaggatcttcttttgagtgcaattccctaaaaccagtactctaatagtttttcctagaacacctagatccttttaaattaaaaatgaagttttaaatcaatctaaagtagtggatctaggaaaatttaatttttacttcaaaatttagttagatttcattatatgagtaaacttggtctgacagttaccaatgcttaatcagtgaggcaatatactcatttgaaccagactgtcaatggttacgaattagtcactccgtcctatctcagcgatctgtctatttcgttcatccatagttgcctgactcccggatagagtcgctagacagataaagcaagtaggtatcaacggactgagggcgtcgtgtagataactacgatacgggagggcttaccatctggccccagtggcagcacatctattgatgctatgccctcccgaatggtagaccggggtcacctgcaatgataccgcgagacccacgctcaccggctccagatttatcagcagacgttactatggcgctctgggtgcgagtggccgaggtctaaatagtcgtataaaccagccagccggaagggccgagcgcagaagtggtcctgcaacttttatttggtcggtcggccttcccggctcgcgtcttcaccaggacgttgaaaatccgcctccatccagtctattaattgttgccgggaagctagagtaagtataggcggaggtaggtcagataattaacaacggcccttcgatctcattcatgttcgccagttaatagtttgcgcaacgttgttgccattgctacaggcatccaagcggtcaattatcaaacgcgttgcaacaacggtaacgatgtccgtaggtggtgtcacgctcgtcgtttggtatggcttcattcagctccggttcccacaccacagtgcgagcagcaaaccataccgaagtaagtcgaggccaagggtacgatcaaggcgagttacatgatcccccatgttgtgcaaaaaagcggttatgctagttccgctcaatgtactagggggtacaacacgttttttcgccaatgctccttcggtcctccgatcgttgtcagaagtaagttggccgcagtgttacgaggaagccaggaggctagcaacagtcttcattcaaccggcgtcacaattcactcatggttatggcagcactgcataattctcttactgtcatgccatcagtgagtaccaataccgtcgtgacgtattaagagaatgacagtacggtagcgtaagatgcttttctgtgactggtgagtactcaaccaagtcattctgaggcattctacgaaaagacactgaccactcatgagttggttcagtaagactcaatagtgtatgcggcgaccgagttgctcttgcccggcgtcaatacgggatttatcacatacgccgctggctcaacgagaacgggccgcagttatgccctaaataccgcgccacatagcagaactttaaaagtgctcatcattggaaaacgttatggcgcggtgtatcgtcttgaaattttcacgagtagtaaccttttgcttcttcggggcgaaaactctcaaggatcttaccgctgttgagatccagttaagaagccccgcttttgagagttcctagaatggcgacaactctaggtcaacgatgtaacccactcgtgcacccaactgatcttcagcatcttttactttcgctacattgggtgagcacgtgggttgactagaagtcgtagaaaatgaaagaccagcgtttctgggtgagcaaaaacaggaaggcaaaatgccgcaaaaaatggtcgcaaagacccactcgtttttgtccttccgttttacggcgttttttgggaataagggcgacacggaaatgttgaatactcatactcttcctttttccccttattcccgctgtgcctttacaacttatgagtatgagaaggaaaaagaatattattgaagcatttatcagggttattgtctcatgagcggatacatattataataacttcgtaaatagtcccaataacagagtactcgcctatgtattttgaatgtatttagaaaaataaacaaataggggttccgcgcacatttccaaacttacataaatctttttatttgtttatccccaaggcgcgtgtaaaggccgaaaagtgccacctgacgtctaagaaaccattattatcatgacattaaggcttttcacggtggactgcagattctttggtaataatagtactgtaattcctataaaaataggcgtatcacgaggccctttcgtcggatatttttatccgcatagtgctccgggaaagcag

SEQ ID NO: 30 - DNA, 8408 bp, Artificial sequence
  Description of Artificial Sequence pIVEX-2.8 CAT mut AviTagTM; vector with coding sequence for mutated AviTagTM fused to chloramphenicol acetyl transferase (CAT)
tcgcgcgtttcggtgatgacggtgaaaacctctgacacatgcagctcccgagcgcgcaaagccactactgccacttttggagactgtgtacgtcgagggcgagacggtcacagcttgtctgtaagcggatgccgggagcagacaagcccgctctgccagtgtcgaacagacattcgcctacggccctcgtctgttcgggctcagggcgcgtcagcgggtgttggcgggtgtcggggctggcttaactatgagtcccgcgcagtcgcccacaaccgcccacagccccgaccgaattgataccggcatcagagcagattgtactgagagtgcaccatatatgcggtgtgaaagccgtagtctcgtctaacatgactctcacgtggtatatacgccacacttttaccgcacagatgcgtaaggagaaaataccgcatcaggcgccattcgccaatggcgtgtctacgcattcctcttttatggcgtagtccgcggtaagcggtttcaggctgcgcaactgttgggaagggcgatcggtgcgggcctcttcgctaagtccgacgcgttgacaacccttcccgctagccacgcccggagaagcgaattacgccagctggcgaaagggggatgtgctgcaaggcgattaagttgggtaatgcggtcgaccgctttccccctacacgacgttccgctaattcaaccctaacgccagggttttcccagtcacgacgttgtaaaacgacggccagtgccattgcggtcccaaaagggtcagtgctgcaacattttgctgccggtcacggaagcttgcatgcaaggagatggcgcccaacagtcccccggccacggggccttcgaacgtacgttcctctaccgcgggttgtcagggggccggtgccccggtgccaccatacccacgccgaaacaagcgctcatgagcccgaagtggcgagacggtggtatgggtgcggctttgttcgcgagtactcgggcttcaccgctccccgatcttccccatcggtgatgtcggcgatataggcgccagcaaccgcagggctagaaggggtagccactacagccgctatatccgcggtcgttggcgtcctgtggcgccggtgatgccggccacgatgcgtccggcgtagaggatcgaggacaccgcggccactacggccggtgctacgcaggccgcatctcctagctgatctcgatcccgcgaaattaatacgactcactatagggagaccacaacgctagagctagggcgctttaattatgctgagtgatatccctctggtgttgcgtttccctctagaaataattttgtttaactttaagaaggagatataccatcaaagggagatctttattaaaacaaattgaaattcttcctctatatggtagagtggtttaaacgatattttcgaggctcagaaaatcgaatggcacgaaactcaccaaatttgctataaaagctccgagtcttttagcttaccgtgcttttcgaaggccgcggccgcttaattaaacatatgaccatggagaaaaaaatcagcttccggcgccggcgaattaatttgtatactggtacctctttttttagactggatataccaccgttgatatatcccaatggcatcgtaaagaacattttgacctatatggtggcaactatatagggttaccgtagcatttcttgtaaatgaggcatttcagtcagttgctcaatgtacctataaccagaccgttcagcactccgtaaagtcagtcaacgagttacatggatattggtctggcaagtcgtggatattacggcctttttaaagaccgtaaagaaaaataagcacaagtttacctataatgccggaaaaatttctggcatttctttttattcgtgttcaaatatccggcctttattcacattcttgcccgcctgatgaatgctcatccggaataggccggaaataagtgtaagaacgggcggactacttacgagtaggcctactccgtatggcaatgaaagacggtgagctggtgatatgggatagtgttctgaggcataccgttactttctgccactcgaccactataccctatcacaagacccttgttacaccgttttccatgagcaaactgaaacgttttcatcgctctgggaacaatgtggcaaaaggtactcgtttgactttgcaaaagtagcgagtggagtgaataccacgacgatttccggcagtttctacacatatattcgcaacctcacttatggtgctgctaaaggccgtcaaagatgtgtatataagcgtagatgtggcgtgttacggtgaaaacctggcctatttccctaaagggtttatctacaccgcacaatgccacttttggaccggataaagggatttcccaaatttgagaatatgtttttcgtctcagccaatccctgggtgagtttcaccagtaactcttatacaaaaagcagagtcggttagggacccactcaaagtggtcatttgatttaaacgtggccaatatggacaacttcttcgcccccgttttcacaaactaaatttgcaccggttatacctgttgaagaagcgggggcaaaagtggatgggcaaatattatacgcaaggcgacaaggtgctgatgccgctggcgactacccgtttataatatgcgttccgctgttccacgactacggcgaccgctttcaggttcatcatgccgtttgtgatggcttccatgtcggcagaatgcttaagtccaagtagtacggcaaacactaccgaaggtacagccgtcttacgaaaatgaattacaacagtactgcgatgagtggcagggcggggcgcccgggatttacttaatgttgtcatgacgctactcaccgtcccgccccgcgggccctaccggtaagatccggctgctaacaaagcccgaaaggaagctgagttggctgggccattctaggccgacgattgtttcgggctttccttcgactcaaccgacctgccaccgctgagcaataactagcataaccccttggggcctctaaacgggacggtggcgactcgttattgatcgtattggggaaccccggagatttgccgtcttgaggggttttttgctgaaaggaggaactatatccggatatccacacagaactccccaaaaaacgactttcctccttgatataggcctataggtgtggacgggtgtggtcgccatgatcgcgtagtcgatagtggctccaagtagccctgcccacaccagcggtactagcgcatcagctatcaccgaggttcatcggaagcgagcaggactgggcggcggccaaagcggtcggacagtgctccgagcttcgctcgtcctgacccgccgccggtttcgccagcctgtcacgaggctcaacgggtgcgcatagaaattgcatcaacgcatatagcgctagcagcacgcttgcccacgcgtatctttaacgtagttgcgtatatcgcgatcgtcgtgcgcatagtgactggcgatgctgtcggaatggacgatatcccgcaagaggcccgtatcactgaccgctacgacagccttacctgctatagggcgttctccgggggcagtaccggcataaccaagcctatgcctacagcatccagggtgacggtccgtcatggccgtattggttcggatacggatgtcgtaggtcccactgccagccgaggatgacgatgagcgcattgttagatttcatacacggtgcctgaccggctcctactgctactcgcgtaacaatctaaagtatgtgccacggactgtgcgttagcaatttaactgtgataaactaccgcattaaagcttatcgatgacgcaatcgttaaattgacactatttgatggcgtaatttcgaatagctacataagctgtcaaacatgagaattcgtaatcatgtcatagctgtttcctgttattcgacagtttgtactcttaagcattagtacagtatcgacaaaggacagtgaaattgttatccgctcacaattccacacaacatacgagccggaagcacactttaacaataggcgagtgttaaggtgtgttgtatgctcggccttcgttaaagtgtaaagcctggggtgcctaatgagtgagctaactcacattaattatttcacatttcggaccccacggattactcactcgattgagtgtaattaagcgttgcgctcactgcccgctttccagtcgggaaacctgtcgtgccagctcgcaacgcgagtgacgggcgaaaggtcagccctttggacagcacggtcgagcattaatgaatcggccaacgcgcggggagaggcggtttgcgtattgggccgtaattacttagccggttgcgcgcccctctccgccaaacgcataacccggctcttccgcttcctcgctcactgactcgctgcgctcggtcgttcggctgcgagaaggcgaaggagcgagtgactgagcgacgcgagccagcaagccgaccggcgagcggtatcagctcactcaaaggcggtaatacggttatccacagagccgctcgccatagtcgagtgagtttccgccattatgccaataggtgtctatcaggggataacgcaggaaagaacatgtgagcaaaaggccagcaaaaggtagtcccctattgcgtcctttcttgtacactcgttttccggtcgttttccccaggaaccgtaaaaaggccgcgttgctggcgtttttccataggctccgcggtccttggcatttttccggcgcaacgaccgcaaaaaggtatccgaggcgccccctgacgagcatcacaaaaatcgacgctcaagtcagaggtggcgaaagggggactgctcgtagtgtttttagctgcgagttcagtctccaccgctttcccgacaggactataaagataccaggcgtttccccctggaagctccctcggggctgtcctgatatttctatggtccgcaaagggggaccttcgagggagctgcgctctcctgttccgaccctgccgcttaccggatacctgtccgcctttacgcgagaggacaaggctgggacggcgaatggcctatggacaggcggaaactcccttcgggaagcgtggcgctttctcatagctcacgctgtaggtatctgagggaagcccttcgcaccgcgaaagagtatcgagtgcgacatccatagacagttcggtgtaggtcgttcgctccaagctgggctgtgtgcacgaaccccgtcaagccacatccagcaagcgaggttcgacccgacacacgtgcttggggccgttcagcccgaccgctgcgccttatccggtaactatcgtcttgagtccggcaagtcgggctggcgacgcggaataggccattgatagcagaactcaggaacccggtaagacacgacttatcgccactggcagcagccactggtaacagttgggccattctgtgctgaatagcggtgaccgtcgtcggtgaccattgtcgattagcagagcgaggtatgtaggcggtgctacagagttcttgaagtggtctaatcgtctcgctccatacatccgccacgatgtctcaagaacttcaccaggcctaactacggctacactagaaggacagtatttggtatctgcgctctgccggattgatgccgatgtgatcttcctgtcataaaccatagacgcgagacctgaagccagttaccttcggaaaaagagttggtagctcttgatccggcaagacttcggtcaatggaagcctttttctcaaccatcgagaactaggccgttacaaaccaccgctggtagcggtggtttttttgtttgcaagcagcagattatgtttggtggcgaccatcgccaccaaaaaaacaaacgttcgtcgtctaatcgcgcagaaaaaaaggatctcaagaagatcctttgatcttttctacgggggcgcgtctttttttcctagagttcttctaggaaactagaaaagatgcccctctgacgctcagtggaacgaaaactcacgttaagggattttggtcatgagagactgcgagtcaccttgcttttgagtgcaattccctaaaaccagtactcattatcaaaaaggatcttcacctagatccttttaaattaaaaatgaagtttaatagtttttcctagaagtggatctaggaaaatttaatttttacttcaattaaatcaatctaaagtatatatgagtaaacttggtctgacagttaccaaaatttagttagatttcatatatactcatttgaaccagactgtcaatggtttgcttaatcagtgaggcacctatctcagcgatctgtctatttcgttcatcacgaattagtcactccgtggatagagtcgctagacagataaagcaagtagcatagttgcctgactccccgtcgtgtagataactacgatacgggagggctgtatcaacggactgaggggcagcacatctattgatgctatgccctcccgataccatctggccccagtgctgcaatgataccgcgagacccacgctcaccgatggtagaccggggtcacgacgttactatggcgctctgggtgcgagtggcgctccagatttatcagcaataaaccagccagccggaagggccgagcgcagcgaggtctaaatagtcgttatttggtcggtcggccttcccggctcgcgtcaagtggtcctgcaactttatccgcctccatccagtctattaattgttgccttcaccaggacgttgaaataggcggaggtaggtcagataattaacaacgggggaagctagagtaagtagttcgccagttaatagtttgcgcaacgttgttcccttcgatctcattcatcaagcggtcaattatcaaacgcgttgcaacaagccattgctacaggcatcgtggtgtcacgctcgtcgtttggtatggcttccggtaacgatgtccgtagcaccacagtgcgagcagcaaaccataccgaagattcagctccggttcccaacgatcaaggcgagttacatgatcccccatgttaagtcgaggccaagggttgctagttccgctcaatgtactagggggtacatgtgcaaaaaagcggttagctccttcggtcctccgatcgttgtcagaagtacacgttttttcgccaatcgaggaagccaggaggctagcaacagtcttcaaagttggccgcagtgttatcactcatggttatggcagcactgcataattcttcaaccggcgtcacaatagtgagtaccaataccgtcgtgacgtattaagtcttactgtcatgccatccgtaagatgcttttctgtgactggtgagtactagaatgacagtacggtaggcattctacgaaaagacactgaccactcatgacaaccaagtcattctgagaatagtgtatgcggcgaccgagttgctcttgcgttggttcagtaagactcttatcacatacgccgctggctcaacgagaacgccggcgtcaatacgggataataccgcgccacatagcagaactttaaaagtggccgcagttatgccctattatggcgcggtgtatcgtcttgaaattttcagctcatcattggaaaacgttcttcggggcgaaaactctcaaggatcttaccgagtagtaaccttttgcaagaagccccgcttttgagagttcctagaatgcgctgttgagatccagttcgatgtaacccactcgtgcacccaactgatctgcgacaactctaggtcaagctacattgggtgagcacgtgggttgactagatcagcatcttttactttcaccagcgtttctgggtgagcaaaaacaggaagagtcgtagaaaatgaaagtggtcgcaaagacccactcgtttttgtccttcgcaaaatgccgcaaaaaagggaataagggcgacacggaaatgttgaataccgttttacggcgttttttcccttattcccgctgtgcctttacaacttatgtcatactcttcctttttcaatattattgaagcatttatcagggttattgtagtatgagaaggaaaaagttataataacttcgtaaatagtcccaataacactcatgagcggatacatatttgaatgtatttagaaaaataaacaaatagggagtactcgcctatgtataaacttacataaatctttttatttgtttatccggttccgcgcacatttccccgaaaagtgccacctgacgtctaagaaaccaccaaggcgcgtgtaaaggggcttttcacggtggactgcagattctttggtttattatcatgacattaacctataaaaataggcgtatcacgaggccctttaataatagtactgtaattggatatttttatccgcatagtgctccgggaaacgtcgcag

SEQ ID NO: 31 - DNA, 8138 bp, Artificial sequence
  Description of Artificial SequencepIVEX-2.8 EPO mut AviTagTM; vector with coding sequence for mutated AviTagTM fused to erythropoetin (EPO)
tcgcgcgtttcggtgatgacggtgaaaacctctgacacatgcagctcccgagcgcgcaaagccactactgccacttttggagactgtgtacgtcgagggcgagacggtcacagcttgtctgtaagcggatgccgggagcagacaagcccgctctgccagtgtcgaacagacattcgcctacggccctcgtctgttcgggctcagggcgcgtcagcgggtgttggcgggtgtcggggctggcttaactatgagtcccgcgcagtcgcccacaaccgcccacagccccgaccgaattgataccggcatcagagcagattgtactgagagtgcaccatatatgcggtgtgaaagccgtagtctcgtctaacatgactctcacgtggtatatacgccacacttttaccgcacagatgcgtaaggagaaaataccgcatcaggcgccattcgccaatggcgtgtctacgcattcctcttttatggcgtagtccgcggtaagcggtttcaggctgcgcaactgttgggaagggcgatcggtgcgggcctcttcgctaagtccgacgcgttgacaacccttcccgctagccacgcccggagaagcgaattacgccagctggcgaaagggggatgtgctgcaaggcgattaagttgggtaatgcggtcgaccgctttccccctacacgacgttccgctaattcaaccctaacgccagggttttcccagtcacgacgttgtaaaacgacggccagtgccattgcggtcccaaaagggtcagtgctgcaacattttgctgccggtcacggaagcttgcatgcaaggagatggcgcccaacagtcccccggccacggggccttcgaacgtacgttcctctaccgcgggttgtcagggggccggtgccccggtgccaccatacccacgccgaaacaagcgctcatgagcccgaagtggcgagacggtggtatgggtgcggctttgttcgcgagtactcgggcttcaccgctccccgatcttccccatcggtgatgtcggcgatataggcgccagcaaccgcagggctagaaggggtagccactacagccgctatatccgcggtcgttggcgtcctgtggcgccggtgatgccggccacgatgcgtccggcgtagaggatcgaggacaccgcggccactacggccggtgctacgcaggccgcatctcctagctgatctcgatcccgcgaaattaatacgactcactatagggagaccacaacgctagagctagggcgctttaattatgctgagtgatatccctctggtgttgcgtttccctctagaaataattttgtttaactttaagaaggagatataccatcaaagggagatctttattaaaacaaattgaaattcttcctctatatggtagagtggtttaaacgatattttcgaggctcagaaaatcgaatggcacgaaactcaccaaatttgctataaaagctccgagtcttttagcttaccgtgcttttcgaaggccgcggccgcttaattaaacatatgaccatggttatcgaaggcagcttccggcgccggcgaattaatttgtatactggtaccaatagcttccgcgcgccccaccacgcctcatctgtgacagccgagtcctggagaggtacctgcgcggggtggtgcggagtagacactgtcggctcaggacctctccatggacttggaggccaaggaggccgagaatatcacgacgggctgtgctgaacactgaacctccggttcctccggctcttatagtgctgcccgacacgacttgtgagcagcttgaatgagaatatcactgtcccagacaccaaagttaatttctatcgtcgaacttactcttatagtgacagggtctgtggtttcaattaaagatagcctggaagaggatggaggtcgggcagcaggccgtagaagtctggcagggcggaccttctcctacctccagcccgtcgtccggcatcttcagaccgtccccctggccctgctgtcggaagctgtcctgcggggccaggccctgttggtcaggaccgggacgacagccttcgacaggacgccccggtccgggacaaccagtactcttcccagccgtgggagcccctgcagctgcatgtggataaagccgtctgagaagggtcggcaccctcggggacgtcgacgtacacctatttcggcagagtggccttcgcagcctcaccactctgcttcgggctctgggagcccagaatcaccggaagcgtcggagtggtgagacgaagcccgagaccctcgggtcttggaagccatctcccctccagatgcggcctcagctgctccactccgaacaaccttcggtagaggggaggtctacgccggagtcgacgaggtgaggcttgtttcactgctgacactttccgcaaactcttccgagtctactccaatttcctcagtgacgactgtgaaaggcgtttgagaaggctcagatgaggttaaaggagcggggaaagctgaagctgtacacaggggaggcctgcaggacaggggacaggcccctttcgacttcgacatgtgtcccctccggacgtcctgtcccctgtcatgataacccgggatccggtaagatccggctgctaacaaagcccgaaaggtactattgggccctaggccattctaggccgacgattgtttcgggctttccaagctgagttggctgctgccaccgctgagcaataactagcataaccccttttcgactcaaccgacgacggtggcgactcgttattgatcgtattggggaaggggcctctaaacgggtcttgaggggttttttgctgaaaggaggaactatccccggagatttgcccagaactccccaaaaaacgactttcctccttgataatccggatatccacaggacgggtgtggtcgccatgatcgcgtagtcgatataggcctataggtgtcctgcccacaccagcggtactagcgcatcagctatgtggctccaagtagcgaagcgagcaggactgggcggcggccaaagcggtccaccgaggttcatcgcttcgctcgtcctgacccgccgccggtttcgccagggacagtgctccgagaacgggtgcgcatagaaattgcatcaacgcatatacctgtcacgaggctcttgcccacgcgtatctttaacgtagttgcgtatatgcgctagcagcacgccatagtgactggcgatgctgtcggaatggacgatacgcgatcgtcgtgcggtatcactgaccgctacgacagccttacctgctattcccgcaagaggcccggcagtaccggcataaccaagcctatgcctacagcagggcgttctccgggccgtcatggccgtattggttcggatacggatgtcgatccagggtgacggtgccgaggatgacgatgagcgcattgttagatttcataggtcccactgccacggctcctactgctactcgcgtaacaatctaaagttacacggtgcctgactgcgttagcaatttaactgtgataaactaccgcatatgtgccacggactgacgcaatcgttaaattgacactatttgatggcgtataaagcttatcgatgataagctgtcaaacatgagaattcgtaatcatgtcatttcgaatagctactattcgacagtttgtactcttaagcattagtacagatagctgtttcctgtgtgaaattgttatccgctcacaattccacacaacatatcgacaaaggacacactttaacaataggcgagtgttaaggtgtgttgttacgagccggaagcataaagtgtaaagcctggggtgcctaatgagtgagcatgctcggccttcgtatttcacatttcggaccccacggattactcactcgtaactcacattaattgcgttgcgctcactgcccgctttccagtcgggaaaattgagtgtaattaacgcaacgcgagtgacgggcgaaaggtcagccctttcctgtcgtgccagctgcattaatgaatcggccaacgcgcggggagaggcgggacagcacggtcgacgtaattacttagccggttgcgcgcccctctccgcgtttgcgtattgggcgctcttccgcttcctcgctcactgactcgctgcgccaaacgcataacccgcgagaaggcgaaggagcgagtgactgagcgacgcgtcggtcgttcggctgcggcgagcggtatcagctcactcaaaggcggtaatagccagcaagccgacgccgctcgccatagtcgagtgagtttccgccattaacggttatccacagaatcaggggataacgcaggaaagaacatgtgagcaatgccaataggtgtcttagtcccctattgcgtcctttcttgtacactcgttaaggccagcaaaaggccaggaaccgtaaaaaggccgcgttgctggcgtttttccggtcgttttccggtccttggcatttttccggcgcaacgaccgcaaattccataggctccgcccccctgacgagcatcacaaaaatcgacgctcaagaaggtatccgaggcggggggactgctcgtagtgtttttagctgcgagttctcagaggtggcgaaacccgacaggactataaagataccaggcgtttccccagtctccaccgctttgggctgtcctgatatttctatggtccgcaaaggggctggaagctccctcgtgcgctctcctgttccgaccctgccgcttaccggagaccttcgagggagcacgcgagaggacaaggctgggacggcgaatggccttacctgtccgcctttctcccttcgggaagcgtggcgctttctcatagctcatggacaggcggaaagagggaagcccttcgcaccgcgaaagagtatcgagacgctgtaggtatctcagttcggtgtaggtcgttcgctccaagctgggcttgcgacatccatagagtcaagccacatccagcaagcgaggttcgacccgagtgtgcacgaaccccccgttcagcccgaccgctgcgccttatccggtaaccacacgtgcttggggggcaagtcgggctggcgacgcggaataggccattgtatcgtcttgagtccaacccggtaagacacgacttatcgccactggcagcatagcagaactcaggttgggccattctgtgctgaatagcggtgaccgtcgagccactggtaacaggattagcagagcgaggtatgtaggcggtgctacagtcggtgaccattgtcctaatcgtctcgctccatacatccgccacgatgtcagttcttgaagtggtggcctaactacggctacactagaaggacagtattttcaagaacttcaccaccggattgatgccgatgtgatcttcctgtcataaaggtatctgcgctctgctgaagccagttaccttcggaaaaagagttggtagccatagacgcgagacgacttcggtcaatggaagcctttttctcaaccatcctcttgatccggcaaacaaaccaccgctggtagcggtggtttttttgtttgagaactaggccgtttgtttggtggcgaccatcgccaccaaaaaaacaaagcaagcagcagattacgcgcagaaaaaaaggatctcaagaagatcctttgcgttcgtcgtctaatgcgcgtctttttttcctagagttcttctaggaaacatcttttctacggggtctgacgctcagtggaacgaaaactcacgttaaggtagaaaagatgccccagactgcgagtcaccttgcttttgagtgcaattccgattttggtcatgagattatcaaaaaggatcttcacctagatccttttaactaaaaccagtactctaatagtttttcctagaagtggatctaggaaaattattaaaaatgaagttttaaatcaatctaaagtatatatgagtaaacttggtaatttttacttcaaaatttagttagatttcatatatactcatttgaacctctgacagttaccaatgcttaatcagtgaggcacctatctcagcgatctgagactgtcaatggttacgaattagtcactccgtggatagagtcgctagactctatttcgttcatccatagttgcctgactccccgtcgtgtagataactaagataaagcaagtaggtatcaacggactgaggggcagcacatctattgatcgatacgggagggcttaccatctggccccagtgctgcaatgataccgcgagctatgccctcccgaatggtagaccggggtcacgacgttactatggcgctgacccacgctcaccggctccagatttatcagcaataaaccagccagccggctgggtgcgagtggccgaggtctaaatagtcgttatttggtcggtcggccaagggccgagcgcagaagtggtcctgcaactttatccgcctccatccagtttcccggctcgcgtcttcaccaggacgttgaaataggcggaggtaggtcactattaattgttgccgggaagctagagtaagtagttcgccagttaatagtgataattaacaacggcccttcgatctcattcatcaagcggtcaattatcattgcgcaacgttgttgccattgctacaggcatcgtggtgtcacgctcgtcaacgcgttgcaacaacggtaacgatgtccgtagcaccacagtgcgagcaggtttggtatggcttcattcagctccggttcccaacgatcaaggcgagttacaaaccataccgaagtaagtcgaggccaagggttgctagttccgctcaatcatgatcccccatgttgtgcaaaaaagcggttagctccttcggtcctccggtactagggggtacaacacgttttttcgccaatcgaggaagccaggaggcatcgttgtcagaagtaagttggccgcagtgttatcactcatggttatggctagcaacagtcttcattcaaccggcgtcacaatagtgagtaccaataccgagcactgcataattctcttactgtcatgccatccgtaagatgcttttctgtcgtgacgtattaagagaatgacagtacggtaggcattctacgaaaagactgactggtgagtactcaaccaagtcattctgagaatagtgtatgcggcgaactgaccactcatgagttggttcagtaagactcttatcacatacgccgctccgagttgctcttgcccggcgtcaatacgggataataccgcgccacatagggctcaacgagaacgggccgcagttatgccctattatggcgcggtgtatccagaactttaaaagtgctcatcattggaaaacgttcttcggggcgaaaacgtcttgaaattttcacgagtagtaaccttttgcaagaagccccgcttttgtctcaaggatcttaccgctgttgagatccagttcgatgtaacccactcgtagagttcctagaatggcgacaactctaggtcaagctacattgggtgagcagcacccaactgatcttcagcatcttttactttcaccagcgtttctgggtgcgtgggttgactagaagtcgtagaaaatgaaagtggtcgcaaagacccacagcaaaaacaggaaggcaaaatgccgcaaaaaagggaataagggcgacactcgtttttgtccttccgttttacggcgttttttcccttattcccgctgtgggaaatgttgaatactcatactcttcctttttcaatattattgaagcattcctttacaacttatgagtatgagaaggaaaaagttataataacttcgtaatatcagggttattgtctcatgagcggatacatatttgaatgtatttagaaatagtcccaataacagagtactcgcctatgtataaacttacataaatcttaaataaacaaataggggttccgcgcacatttccccgaaaagtgccacctgtttatttgtttatccccaaggcgcgtgtaaaggggcttttcacggtggacacgtctaagaaaccattattatcatgacattaacctataaaaataggcgttgcagattctttggtaataatagtactgtaattggatatttttatccgcaatcacgaggccctttcgtctagtgctccgggaaagcag

(19) United States
(12) Patent Application Publication
(10) Pub. No.: US 2006/0046285 A1
(43) Pub. Date: Mar. 2, 2006

(54) Enhanced expression of fusion polypeptides with a biotinylation tag

(76) Inventors: Manfred Watzele, Weilheim (DE); Dieter Voges, Muenchen (DE); Frank Wedekind, Benediktbeuern (DE)

Correspondence Address: Roche Diagnostics Corporation, 9115 Hague Road, PO Box 50457, Indianapolis, IN, 46250-0457, US

(21) Appl. No.: 11/211,339

(22) Filed: Aug. 25, 2005

Related U.S. Application Data
(63) Continuation of application No. PCT/EP04/01973, filed on Feb. 27, 2004.

(30) Foreign Application Priority Data
Feb. 28, 2003 (EP) EP 03004326.9

(51) Int. Cl. C12P 21/04; C12N 9/10; C07H 21/04

(52) U.S. Cl. 435/69.7; 435/320.1; 435/193; 435/325; 536/23.2

(57) ABSTRACT

The invention provides the means to enhance in E. coli-based expression systems the formation of fusion polypeptides containing as an N-terminal tag a biotinylation polypeptide. By way of specifically exchanging in the nucleic acid sequence encoding the biotinylation polypeptide nucleotides at 11 discrete positions enhances the formation of the total fusion polypeptide by at least 40%.

### RELATED APPLICATIONS

This application is a continuation of international patent application PCT/EP2004/001973 filed Feb. 27, 2004, which claims priority to European patent application EP 03004326.9 filed Feb. 28, 2003.

### FIELD OF THE INVENTION

The present invention relates to nucleic acids encoding a polypeptide capable of being biotinylated by holocarboxylase synthetase. In particular, the present invention relates to the formation of fusion polypeptides comprising an N-terminal polypeptide capable of being biotinylated by holocarboxylase synthetase and a C-terminal polypeptide with a biological function. More particularly, the invention relates to the enhanced formation of such fusion polypeptides by means of expression in vitro or in vivo E. coli-based expression systems. The invention therefore relates to the field of molecular biology, but given the diverse uses for recombinant proteins, the invention also relates to the fields of chemistry, pharmacology, biotechnology, and medical diagnostics.

### BACKGROUND OF THE INVENTION

The enzyme holocarboxylase synthetase of E. coli (BirA, a biotin ligase) catalyzes in vivo the biotinylation, that is the covalent addition of biotin to the ε-amino group of a lysine side chain in its natural substrate, biotin carboxyl carrier protein (BCCP) (Cronan, J. E., Jr., et al., J. Biol. Chem. 265 (1990) 10327-10333). In E. coli only BCCP is biotinylated. This protein is a subunit of acetyl-CoA carboxylase. The reaction is catalysed by the biotin-protein ligase, the product of the BirA gene (Cronan, J. E., Jr., Cell 58 (1989) 427-429).

A BirA substrate consisting of a sequence of 13 amino acids was defined as a biotinylation polypeptide in fusion polypeptides (Schatz, P. J., Biotechnology 11 (1993) 1138-1143). WO 95/04069 describes biotinylation peptides that can be fused to other peptides or proteins of interest using recombinant DNA techniques. The resulting fusion polypeptides can be biotinylated in vivo or in vitro by BirA holocarboxylase synthetase. Particularly WO 95/04069 describes the expression of such fusion polypeptides in E. coli and anticipates expression in cell-free expression systems. But both documents are completely silent regarding the impact of the nucleic acid sequence that is encoding an N-terminal biotinylation polypeptide on the expressed quantity of the fusion polypeptide.

U.S. Pat. Nos. 5,723,584, 5,874,239, 5,932,433 and 6,265,552 provide further amino acid sequences for biotinylation polypeptides to be used for generating fusions with polypeptides of interest. Regarding N-terminally tagged fusion polypeptides, the documents describe the chemical synthesis of nucleic acid sequences that were biased in order to fit a consensus biotinylation polypeptide sequence. However, the documents are completely silent regarding the impact of the nucleic acid sequence that is encoding an N-terminal biotinylation polypeptide on the expressed quantity of the fusion polypeptide.

The biotinylation polypeptide used in the present invention (SEQ ID NO: 1, AviTag™) is comprised in the pAN-4, pAN-5, pAN-6 series of expression vectors distributed by Avidity Inc., Denver, Colo., USA. The set of 3 different pAN vectors are designed for cloning and expression of N-terminal tagged fusion polypeptides in each reading frame. The DNA sequence encoding the biotinylation polypeptide is the DNA sequence of SEQ ID NO: 3.

Moreover, a synthetic BirA biotinylation polypeptide that was identified by combinatorial methods and consisted of a sequence of 23 amino acids was used to define a minimum sequence required for biotinylation that consisted of a sequence of 14 amino acids (Beckett, D., et al., Protein Sci. 8 (1999) 921-929). The 14-mer was proposed to mimic the acceptor function of BCCP as the natural BirA substrate. The impact of the nucleic acid sequence encoding biotinylation polypeptide on the expressed quantity of the fusion polypeptide was not investigated.

U.S. Pat. No. 6,326,157 describes the construction of fusion polypeptides consisting of green fluorescent protein tagged with a biotinylation polypeptide. However, the document is completely silent regarding the impact of the nucleic acid sequence that is encoding an N-terminal biotinylation polypeptide on the expressed quantity of the fusion polypeptide.

E. coli-based cellular expression systems are well-known to the art and include U.S. Pat. No. 5,232,840 regarding an optimized ribosome-binding site. Particularly cellular E. coli expression systems using the T7 promoter are described in U.S. Pat. Nos. 4,952,496, 5,693,489 and 5,869,320.

Codon usage is one of the best known parameters impacting on the expressed quantity of a polypeptide. Genes in both prokaryotes and eukaryotes show a non-random usage of synonymous codons. The systematic analysis of codon usage patterns in E. coli led to the following observations (de Boer, H. A., and Kastelein, R. A., In: Maximizing gene expression, Reznikoff, W. S., and Gold, L., (eds.), Butterworths, Boston, 1986, pp. 225-285): (1) There is a bias for one or two codons for almost all degenerate codon families. (2) Certain codons are most frequently used by all different genes irrespective of the abundance of the protein. (3) Highly expressed genes exhibit a greater degree of codon bias than do poorly expressed ones. (4) The frequency of use of synonymous codons usually reflects the abundance of their cognate tRNAs. These observations imply that heterologous genes enriched with codons that are rarely used by E. coli may not be expressed efficiently in E. coli.

However, it appears to be difficult to generally and unambiguously predict whether the content of low-usage codons in a specific gene might adversely affect the efficiency of its expression in E. coli. Regarding the efficiency of translation of a polypeptide in E. coli, several influencing factors are superimposed, e.g. positional effects of certain codons, the clustering or interspersion of the rarely used codons, as well as the secondary structure of the mRNA. Nevertheless, from a practical point of view, the codon context of specific genes can have adverse effects on the quantity of expressed polypeptide levels. Usually, this problem is rectified by the alteration of the codons in question, whereby codons in the entire coding sequence are addressed. Another way to address this problem is to co-express the cognate tRNA genes (Makrides, S. C., Microbiol. Rev. 60 (1996) 512-538).

It is also known for in vitro translation systems that adding tRNAs that pair with rarely used codons can increase the expressed quantity of a polypeptide. An example for an in vitro translation system is the RTS 500 System that is distributed by Roche Diagnostics GmbH, Mannheim, Germany (catalogue number 3246817). In this expression system that comprises E. coli lysates, transcription and translation take place simultaneously in a reaction compartment of the reaction device. Substrates and energy components essential for a sustained reaction are continuously supplied via a semipermeable membrane. At the same time, potentially inhibitory reaction by-products are diluted via diffusion through the same membrane into the feeding compartment. Polypeptide is expressed for up to 24 hours yielding up to 5 mg of polypeptide.

Both, for cellular and for cell-free expression systems it is unclear if and to what extent the nucleic acid sequence encoding an N-terminal tag, such as a biotinylation polypeptide, alone can impact on the expressed quantity of a fusion polypeptide. Therefore, the problem to be solved is to provide the means to further enhance in a cell-free as well as in a cellular expression system the formation of a fusion polypeptide that comprises a biotinylation polypeptide.

### SUMMARY OF THE INVENTION

The invention provides the means to enhance in E. coli-based expression systems the formation of fusion polypeptides containing as an N-terminal tag a biotinylation polypeptide. It was surprisingly found that specifically exchanging in the nucleic acid sequence encoding the biotinylation polypeptide nucleotides at 11 discrete positions enhances the formation of the total fusion polypeptide by at least 40%.

Therefore, in a first aspect, the invention provides nucleic acids encoding a polypeptide capable of being biotinylated by holocarboxylase synthetase. In a further aspect, the invention provides an expression vector comprising a nucleic acid according to the invention. In yet a further aspect, the invention provides a method of preparing a biotinylated polypeptide in a cell-free polypeptide synthesis reaction mixture. In yet a further aspect, the invention provides use of a nucleic acid according to the invention for constructing, by way of genetic engineering, a nucleic acid encoding a fusion polypeptide and expressing the same, whereby the fusion polypeptide consists of an N-terminal polypeptide capable of being biotinylated by holocarboxylase synthetase, and a C-terminal polypeptide with a biological function.

### DESCRIPTION OF THE FIGURES

FIG. 1A Coomassie-stained SDS gel. The numbers on the bottom indicate the numbers of the SDS gel lanes. The numbers on the left hand side of the gel indicate molecular weight (given in [kDa]) as indicated by the molecular weight markers to the left of lane 1. In vitro expression (see Example 3) of fusion polypeptides from pIVEX-2.8 CAT WT AviTag with the wildtype sequence encoding the N-terminal tag (lane 1, 5), pIVEX-2.8 CAT mut AviTag with the sequence of SEQ ID NO: 12 encoding the N-terminal tag (lane 2, 6), pIVEX-2.8 EPO WT AviTag with the wildtype sequence encoding the N-terminal tag (lane 3, 7), pIVEX-2.8 EPO mut AviTag with the sequence of SEQ ID NO: 12 encoding the N-terminal tag (lane 4, 8). The total protein suspension of each cell-free polypeptide synthesis reaction mixture was applied in lanes 1-4, the pellet fraction in lanes 5-8.

FIG. 1B Densitometric analysis as described in Example 4 was performed on the areas indicated. The numbers on the bottom indicate the numbers of the SDS gel lanes as in FIG. 1A. It is noted that for the lanes 7 and 8 the numbering of densitometrically quantified bands is changed. Thus, the band designated with “8” is in lane 7 and the band designated with “9” is in lane 8. The values obtained from densitometric quantification are given in Table 1 (Example 4) and are tabulated with reference to the numbering of SDS gel lanes.

FIG. 2 pIVEX-GFP WT AviTag

FIG. 3 pIVEX-2.8 CAT mut AviTag; the site denoted “Xa factor” indicates a cleavage site for factor Xa protease.

FIG. 4 pIVEX-2.8 EPO mut AviTag; the site denoted “Xa factor” indicates a cleavage site for factor Xa protease.

### DETAILED DESCRIPTION OF THE INVENTION

Certain terms are used with particular meaning, or are defined for the first time, in this description of the present invention. For the purposes of the present invention, the following terms are defined by their art-accepted definitions, when such exist, except that when those definitions conflict or partially conflict with the definitions set forth below. In the event of a conflict in definition, the meaning of the terms are first defined by the definitions set forth below.

The term “comprising” is used in the description of the invention and in the claims to mean “including, but not necessarily limited to”.

As used herein, the term “polypeptide with a biological function” refers to a polypeptide which possesses a biological function or activity which is identified through a defined functional assay and which is associated with a particular biologic, morphologic, or phenotypic alteration in a cell or a virus. Examples for polypeptides with a biological function are receptors, transcription factors, kinases, polypeptide subunits of complexes, or antibodies.

The term “polypeptide with a biological function” also encompasses “functional fragments” thereof, thus including all fragments of a the polypeptide with a biological function that retain an activity of the polypeptide. Functional fragments, for example, can vary in size from a polypeptide fragment as small as, e.g., an epitope capable of binding an antibody molecule to a large polypeptide capable of participating in the characteristic induction or programming of phenotypic changes within a cell.

Minor modifications of the primary amino acid sequences of a “polypeptide with a biological function” may result in polypeptides which have substantially equivalent activity as compared to the unmodified counterpart polypeptide. Such modifications may be deliberate, as by site-directed mutagenesis, or may be spontaneous. Further, C- or N-terminal addition of one or more amino acids, insertion of one or more amino acids, as well as deletion of one or more amino acids can also result in a modification of the structure of the resultant molecule without significantly altering its activity. All of the polypeptides produced by these modifications are included under the term “polypeptide with a biological function” as long as the biological activity of the polypeptide still exists.

Additionally, the term “polypeptide with a biological function” encompasses a hybrid polypeptide, that is to say a fusion of two or more polypeptides with biological functions.

The term “polypeptide” denotes a polymer composed of amino acid monomers joined by peptide bonds. A “peptide bond” is a covalent bond between two amino acids in which the α-amino group of one amino acid is bonded to the α-carboxyl group of the other amino acid. All amino acid or polypeptide sequences, unless otherwise designated, are written from the amino terminus (N-terminus) to the carboxy terminus (C-terminus). Amino acid identification uses the three-letter abbreviations as well as the single-letter alphabet of amino acids, i.e. Asp D Aspartic acid, Ile I Isoleucine, Thr T Threonine, Leu L Leucine, Ser S Serine, Tyr Y Tyrosine, Glu E Glutamic acid, Phe F Phenylalanine, Pro P Proline, His H Histidine, Gly G Glycine, Lys K Lysine, Ala A Alanine, Arg R Arginine, Cys C Cysteine, Trp W Tryptophan, Val V Valine, Gln Q Glutamine, Met M Methionine, Asn N Asparagine.

The term “biotinylation polypeptide” is a “polypeptide capable of being biotinylated by holocarboxylase synthetase”. The amino acid sequence of the biotinylation polypeptide provides a sequence motif containing an acceptor site for “biotinylation”, that is the covalent attachment of a biotin molecule by holocarboxylase synthetase.

As used herein, the term “tagging” or “tagging a target sequence” refers to introducing by recombinant methods a nucleic acid encoding a “tag” such as a biotinylation polypeptide into a polypeptide-encoding nucleic acid, i.e. a “target sequence” so that the recombinant nucleic acid encodes a fusion polypeptide which comprises the tag at its C- or N-terminus.

The term “fusion polypeptide” refers to a polypeptide which has been tagged, e.g. with a biotinylation polypeptide. For example, the amino acid sequence of a fusion polypeptide may comprise the amino acid sequence of the biotinylation polypeptide and the amino acid sequence of a target polypeptide. The target polypeptide itself is a polypeptide with a biological function.

“Nucleic acid” as used herein refers to DNA or RNA which may be single- or double-stranded, and represents the sense strand when single-stranded. Nucleic acids are polymers with nucleotides as monomers. Nucleotides are composed of a phosphate moiety, a sugar moiety (ribose or deoxyribose) and an aglyconic heterocyclic moiety, the so-called nucleobase. In a nucleic acid sequence a single letter defines a nucleotide by its nucleobase, i.e. adenine (A), guanine (G), cytosine (C) and thymine (T) or uracil (U).

Nucleic acids encoding fusion polypeptides can be prepared by chemical methods or by genetic engineering. A fusion polypeptide can be obtained by means of “expression” of a nucleic acid encoding the same, that is as a result of transcription and translation of the nucleic acid.

A nucleic acid is “operably linked” when it is placed into a functional relationship with another nucleic acid. For example, a nucleic acid encoding a biotinylation polypeptide is operably linked to a nucleic acid encoding a polypeptide with a biological function if it results in the expression of a fusion polypeptide capable of being biotinylated; a promoter is operably linked to a coding sequence if it affects the transcription of the sequence; or a ribosome binding site is operably linked to a coding sequence if it is positioned so as to facilitate translation. Generally, operably linked means that the nucleic acids being linked are contiguous and, in the case of a nucleic acid encoding, e.g., a biotinylation polypeptide, contiguous and in reading phase. As for DNA, linking is accomplished by ligation at convenient restriction sites. If such sites do not exist then synthetic oligonucleotide adaptors or linkers are used in accord with conventional practice.

All nucleic acid sequences are written in the direction from the 5′ (stands for prime) end to the 3′ end also referred to as 5′ to 3′. The nucleic acid sequences of the invention that encode a polypeptide of SEQ ID NO: 1 are different from previously published nucleic acid sequences such as SEQ ID NO: 3 because of the degeneracy of the genetic code and encode the same polypeptide. Degenerate code stands for a genetic code in which a particular amino acid can be coded by two or more different codons. Degeneracy occurs because of the fact that of the 64 possible base triplets, 3 are used to code the stop signals, and the other 61 are left to code for only 20 different amino acids.

The term “expression system” is well understood in the art to mean either an in vitro system or a cellular or multicellular organism capable of translating or transcribing and translating nucleotide sequences to produce polypeptides. An example for an in vitro expression system, that is to say a cell-free polypeptide synthesis reaction mixture, is described in Zubay, G., Annu. Rev. Genet. 7 (1973) 267-287. Spirin et al. developed in 1988 a continuous-flow cell-free translation and coupled transcription/translation system in which a relatively high amount of protein synthesis occurs (Spirin, A. S., et al., Science 242 (1988) 1162-1164). Examples of application of such systems are documented by Pratt, J. M., et al., Nucleic Acids Research 9 (1981) 4459-4479, and Pratt et al., In: Transcription and Translation: A Practical Approach, Hames and Higgins (eds.), 1984, pp. 179-209, IRL Press. Further developments of the cell-free protein synthesis are described in U.S. Pat. Nos. 5,478,730, 5,571,690, EP 0932664, WO 99/50436, WO 00/58493, and WO 00/55353. Cellular expression systems that are based on E. coli are described in U.S. Pat. Nos. 5,232,840, 4,952,496, US 5,693,489 and 5,869,320.

In a first aspect, the invention provides a nucleic acid of SEQ ID NO: 2 encoding a polypeptide of SEQ ID NO: 1 capable of being biotinylated by holocarboxylase synthetase, characterized in that said nucleic acid differs from SEQ ID NO: 3 by nucleotide exchanges at 6 or more positions selected from the group consisting of the positions 4, 5, 6, 9, 10, 12, 15, 18, 21, 24 or 30, and said nucleic acid, as compared to SEQ ID NO: 3, enhances the formation of a fusion polypeptide, consisting of an N-terminal polypeptide according to SEQ ID NO: 1 and a C-terminal polypeptide with a biological function, by means of expression from a nucleic acid encoding said fusion polypeptide in a cell-free polypeptide synthesis reaction mixture in that at least 40% more fusion polypeptide is formed, whereby the nucleic acid encoding said fusion polypeptide consists of a nucleic acid encoding said N-terminal polypeptide operably linked to a nucleic acid encoding said C-terminal polypeptide.

In a preferred embodiment of the invention, the nucleic acid that is containing A or T at position 4, C or G at position 5, C or T at position 6, A, C or T at position 9, C or T at position 10, A or G at position 12, C or T at position 15, C or T at position 18, C or T at position 21, C or T at position 24, and A or T at position 30, is characterized in that between 5 and 11 nucleotides at said positions are identical to the nucleotides at the same positions in SEQ ID NO: 4, with the proviso that all nucleotides at said positions are identical to the nucleotides at the same positions in SEQ ID NO: 3 or SEQ ID NO: 4, or 10 nucleotides at said positions except position 9 are identical to the nucleotides at the same positions in SEQ ID NO: 4 or SEQ ID NO: 3, and the nucleotide at position 9 is T.

In another preferred embodiment of the invention, the nucleic acid is characterized in that the nucleic acid is selected from the group consisting of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23 or SEQ ID NO: 24.

Another aspect of the invention is an expression vector comprising a nucleic acid according to the invention.

Yet another aspect of the invention is a method of preparing a biotinylated polypeptide in a cell-free polypeptide synthesis reaction mixture which contains an RNA polymerase, ribosomes, tRNA, ATP, GTP, nucleotides and amino acids, comprising the steps of (a) forming in said reaction mixture a fusion polypeptide, consisting of an N-terminal polypeptide according to SEQ ID NO: 1 and a C-terminal polypeptide with a biological function, by means of expression from a nucleic acid consisting of a nucleic acid according to any of the claims 1 to 3 operably linked to a nucleic acid encoding the C-terminal polypeptide; (b) biotinylating said fusion polypeptide in the presence of biotin and holocarboxylase synthetase; (c) isolating said biotinylated fusion polypeptide from said mixture; or incubating said mixture with immobilized avidin or streptavidin under such conditions that said biotinylated fusion polypeptide is bound to said immobilized avidin or streptavidin.

A preferred RNA polymerase is a DNA-dependent RNA polymerase. A very much preferred RNA polymerase is T7 RNA polymerase.

Holocarboxylase synthetase (EC 6.3.4.15, biotin protein ligase, BirA) is an enzyme that catalyses in E. coli the covalent attachment of biotin to its natural substrate, that is BCCP. Biotin ligase is highly specific and reacts only on biotinylation polypeptides showing a very high degree of conservation in the primary structure of the biotin attachment domain. This domain includes preferably the highly conserved AMKM tetrapeptide (Chapman-Smith, A., and Cronan, J. E., Jr., J. Nutr. 129, 2S Suppl., (1999) 477S-484S). Recombinant BirA enzyme is described in WO 99/37785. In order to biotinylate fusion polypeptides, holocarboxylase synthetase can be added to an in vitro expression system as an active enzyme or can be added as a nucleic acid (in an expression vector, e.g. RNA, DNA) which is expressed (transcribed/translated) in the system like the fusion polypeptide.

Therefore, in a preferred embodiment of the invention, the method is characterized in that the reaction mixture contains a nucleic acid encoding holocarboxylase synthetase according to SEQ ID NO: 25 that is expressed in said reaction mixture to provide holocarboxylase synthetase polypeptide. If added as an active enzyme, it is used preferably in an amount of about 10,000 to 15,000 units, preferably 12,500 units. A preferred active enzyme (EC 6.3.4.15) is supplied by Avidity Inc. (Denver, Colo., USA).

In another preferred embodiment of the invention, the method is characterized in that the reaction mixture contains a nucleic acid encoding holocarboxylase synthetase according to SEQ ID NO: 25 that is expressed in the reaction mixture to provide holocarboxylase synthetase polypeptide. The amount of nucleic acid depends on the expression rate of the used vector and the necessary amount of BirA enzyme in the reaction mixture. 1 ng of BirA plasmid DNA (e.g. on the basis of a commercially available E. coli expression vector such as pIVEX vectors, supplied by Roche Diagnostics GmbH, Mannheim, Germany; http://www.biochem.roche.com/RTS), or even less, is sufficient for a quantitative biotinylation reaction of the tagged fusion polypeptides. The maximum yield of expressed and specifically biotinylated fusion polypeptide is achieved, when the desired fusion polypeptide-encoding plasmid DNA is added at 10-15 μg and the plasmid DNA, being responsible for the coexpression of BirA, is introduced with an amount between 1 - 10 ng. The ratio of fusion polypeptide-encoding plasmid DNA to BirA-encoding plasmid DNA was found to be optimal at a ratio of about 1500:1. It was found that the same level as above is sufficient for quantitative biotinylation of the expressed fusion protein. D(+)-biotin was added at 1 to 10 μM, preferably in about 2 μM to the reaction mixture.

After the expression of the fusion polypeptide in the cell-free expression system, biotinylation occurs under standard reaction conditions, preferably within 10 to 30 hours at 20° C. to 36° C., most preferably at about 30° C., and the reaction mixture is preferably, after dialysis, for concentration and buffer exchange, centrifuged.

In a preferred embodiment of the invention, the solution is, due to its high purity, directly used for immobilization of the fusion polypeptide on surfaces which contain immobilized avidin or streptavidin (e.g. microtiter plates or biosensors) without further purification.

According to the invention it is possible to produce highly pure biotinylated polypeptides which can be bound to surfaces in ligand binding experiments, e.g. surface plasmon resonance spectroscopy or ELISA assays.

If required, biotinylated polypeptides produced according to the present invention can be purified further under native conditions using matrices containing immobilized (preferably monomeric) avidin, streptavidin, or derivatives thereof. A variety of useful physically (Kohanski, R. A., and Lane, M. D., Methods Enzymol. 184 (1990) 194-200), chemically (Morag, E., et al., Anal. Biochem. 243 (1996) 257-263) and genetically (Sano, T., and Cantor, C. R., Proc. Natl. Acad. Sci. USA 92 (1995) 3180-3184) modified forms of avidin or streptavidin have been described that still bind biotin specifically but with weaker affinity to facilitate a one step purification procedure.

Yet another aspect of the invention is the use of a nucleic acid according to the invention for constructing, by way of genetic engineering, a nucleic acid encoding a fusion polypeptide, whereby the fusion polypeptide consists of an N-terminal polypeptide of SEQ ID NO: 1 and a C-terminal polypeptide with a biological function. Methods for constructing by way of genetic engineering are well known to the art and are described, in e.g. Sambrook, Fritsch & Maniatis, Molecular Cloning, A Laboratory Manual, 3rd edition, CSHL Press, 2001.

Yet another aspect of the invention is the use of a nucleic acid according to the invention for expressing a fusion polypeptide, whereby the fusion polypeptide consists of an N-terminal polypeptide of SEQ ID NO: 1 and a C-terminal polypeptide with a biological function.

A preferred embodiment of the invention is the use characterized in that the fusion polypeptide is expressed in a cell-free polypeptide synthesis reaction mixture. A preferred cell-free polypeptide synthesis reaction mixture is the RTS 500 in vitro expression system supplied by Roche Diagnostics GmbH (Mannheim, Germany; catalogue number 3246817).

Another preferred embodiment of the invention is the use characterized in that the fusion polypeptide is expressed in E. coli. A preferred E. coli strain is a BL21 (DE3) strain. Even more preferred is a BL21 (DE3) LysS strain. These strains express an active T7 RNA polymerase. Such a strain can be used to transcribe a gene carried by an expression vector, whereby the vector comprises, e.g., a nucleic acid encoding a fusion polypeptide that is operably linked to the T7 promoter. Examples for vectors that have incorporated the T7 promoter and that are capable of being transcribed in the BL21 (DE3) strain or the BL21 (DE3) LysS strain of E. coli are pET vectors (Novagen Inc., Madison, Wis., USA) or pIVEX vectors (Roche Diagnostics GmbH, Mannheim, Germany). Methods for expressing fusion polypeptides are well known to the art and are described (e.g. in: Sambrook, Fritsch & Maniatis, Molecular Cloning, A Laboratory Manual, 3rd edition, CSHL Press, 2001. Also in: Gu, J., et al., Biotechniques 17 (1994) 257, 260, 262).

The following examples, references, sequence listing and figures are provided to aid the understanding of the present invention, the true scope of which is set forth in the appended claims. It is understood that modifications can be made in the procedures set forth without departing from the spirit of the invention.

### EXAMPLE 1

### Mutant Variants of the DNA Sequence Encoding the AviTag Biotinylation Polypeptide

The AviTag biotinylation polypeptide comprises a sequence of 15-17 amino acid residues and can be used as a tag in fusion polypeptides. The AviTag is capable of being biotinylated at a lysine residue by a biotin protein ligase such as the polypeptide encoded by the E. coli BirA gene (Murtif, V. L., and Samols, D., J. Biol. Chem. 262 (1987) 11813-11816). The AviTag biotinylation polypeptide used for the present invention is represented by SEQ ID NO: 1. A DNA sequence encoding the AviTag and expression vectors in which the DNA sequence is incorporated are commercially available from Avidity Inc. (Denver, Colo., USA). The original DNA sequence of which variants were generated is SEQ ID NO: 3. This sequence is also referred to as “wildtype sequence” or “wildtype DNA sequence”.

For the purpose of generating optimized mutant variants of the AviTag encoding DNA sequence, that is to say variants that enhance the expression of a fusion polypeptide that comprises the AviTag biotinylation polypeptide, the wildtype DNA sequence was placed in-frame in front of the test protein green fluorescent protein (GFP; Crameri., A., et al., Nat. Biotechnol. 14 (1996) 315-319) by using conventional cloning methods (Sambrook, Fritsch & Maniatis, Molecular Cloning, A Laboratory Manual, 3rd edition, CSHL Press, 2001). To create mutant sequences of the first ten codons of the wildtype sequence the following two sets of degenerated oligonucleotides were synthesized. The mutated sequences that were synthesized exploited the codon usage for each amino acid without changing the primary sequence. The bases that were changed are indicated in SEQ ID NO: 26 and SEQ ID NO: 27 using the following code: N=any base, Y=pyrimidine (C or T), R=purine (G or A), H=not G (i.e. A, T or C). Thus, two sets of forward primers were generated of which the respective consensus sequences are given in SEQ ID NO: 26 and SEQ ID NO: 27. Each set represented a mixture of primer molecules that essentially represented the possible combinations as defined by the bases that were changed.

In combination with the reverse primer according to SEQ ID NO: 28 that was selected to match an internal sequence of the GFP gene, a PCR reaction was made with the pIVEX-GFP WT AviTag (SEQ ID NO: 29) vector as template. Using the restriction enzymes XbaI and NcoI the PCR products were cleaved, firstly at the XbaI site in the forward primer and secondly at the NcoI site in the reverse primer. In parallel, the pIVEX-GFP WT AviTag vector was cleaved with the same restriction enzymes and the vector fragment was isolated. Subsequently, the cleaved fragments were inserted into the pIVEX-GFP AviTag vector fragments.

The plasmids were ligated and subsequently transformed into a BL21 (DE3) LysS strain of E. coli (Novagen Inc., Madison, Wis., USA) and plated out on LB medium with ampicillin (100 μg/ml), chloramphenicol (100 μg/ml) and IPTG (0.2 mM). After one day of growth bacterial colonies were screened under UV light for GFP expression. The colonies with the brightest fluorescence as judged by visual inspection were picked and plasmids from these colonies were isolated. The AviTag-encoding DNA of these plasmids was subjected to sequence analysis. The screening procedure resulted in a number of mutant variants of the wildtype sequence encoding the AviTag, whereby these variants stimulated a visibly increased GFP signal as compared to the signal of control transformants expressing the pIVEX-GFP WT AviTag vector.

The mutant variants of the wildtype sequence, i.e. DNA sequences encoding a polypeptide of SEQ ID NO: 1 capable of being biotinylated by holocarboxylase synthetase, are represented in SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, and SEQ ID NO: 24.

### EXAMPLE 2

### Comparison of the Mutant Variants of the DNA Sequence Encoding the AviTag Biotinylation Polypeptide and the Wildtype Sequence

The wildtype sequence was compared with SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, and SEQ ID NO: 24, and a consensus sequence was derived for the mutant variants.

Accordingly, the consensus DNA sequence encoding SEQ ID NO: 1 was found to differ from the wildtype sequence, that is the sequence according to SEQ ID NO: 3, by nucleotide exchanges at 6 or more positions selected from the group consisting of the positions 4, 5, 6, 9, 10, 12, 15, 18, 21, 24 or 30.

Furthermore, the consensus DNA sequence was found to contain A or T at position 4, C or G at position 5, C or T at position 6, A, C or T at position 9, C or T at position 10, A or G at position 12, C or T at position 15, C or T at position 18, C or T at position 21, C or T at position 24, and A or T at position 30. The consensus sequence is given in SEQ ID NO: 2.

Furthermore, between 5 and 11 nucleotides at said positions were found to be identical to the nucleotides at the same positions in SEQ ID NO: 4, with the proviso that all nucleotides at said positions were found to be identical to the nucleotides at the same positions in SEQ ID NO: 3 or SEQ ID NO: 4, or 10 nucleotides at said positions except position 9 were found to be identical to the nucleotides at the same positions in SEQ ID NO: 4 or SEQ ID NO: 3, and the nucleotide at position 9 was then found to be T.

### EXAMPLE 3

### Construction of Fusion Polypeptides Using a Mutant Variant of the DNA Sequence Encoding the AviTag Biotinylation Polypeptide

The mutated AviTag sequence according to SEQ ID NO: 12 was inserted in-frame in front of the chloramphenicol acetyl transferase (CAT) gene and the erythropoietin (EPO) gene by way of a PCR cloning approach analogous to the approach described in Example 1. As a result, the plasmids pIVEX-2.8 CAT mut AviTag and pIVEX-2.8 EPO mut AviTag were generated. In addition, the control plasmids pIVEX-2.8 CAT WT AviTag and pIVEX-2.8 EPO WT AviTag were generated that differed from pIVEX-2.8 CAT mut AviTag and pIVEX-2.8 EPO mut AviTag in that the wildtype AviTag sequence, i.e. SEQ ID NO: 3 replaced SEQ ID NO: 12.

All four of these plasmids, i.e. those containing the mutant variants as well as the wildtype controls, were then used for a polypeptide synthesis reaction using the RTS 500 HY Kit (Roche Diagnostics GmbH, Mannheim, Germany) as an in-vitro expression system. Each plasmid was used for a separate in-vitro expression. The polypeptide synthesis reactions were performed identically and in line with the instructions of the supplier. After the reactions were ended, 0.5 μl aliquots of each reaction mixture were directly applied on an SDS-PAGE gel. Another aliquot of each reaction was centrifuged for 15. min at 30,000×g. The supernatants were removed and the pellet fractions were resuspended in the original volume in SDS sample buffer. Again 0.5 μl were applied on the same SDS Page gel.

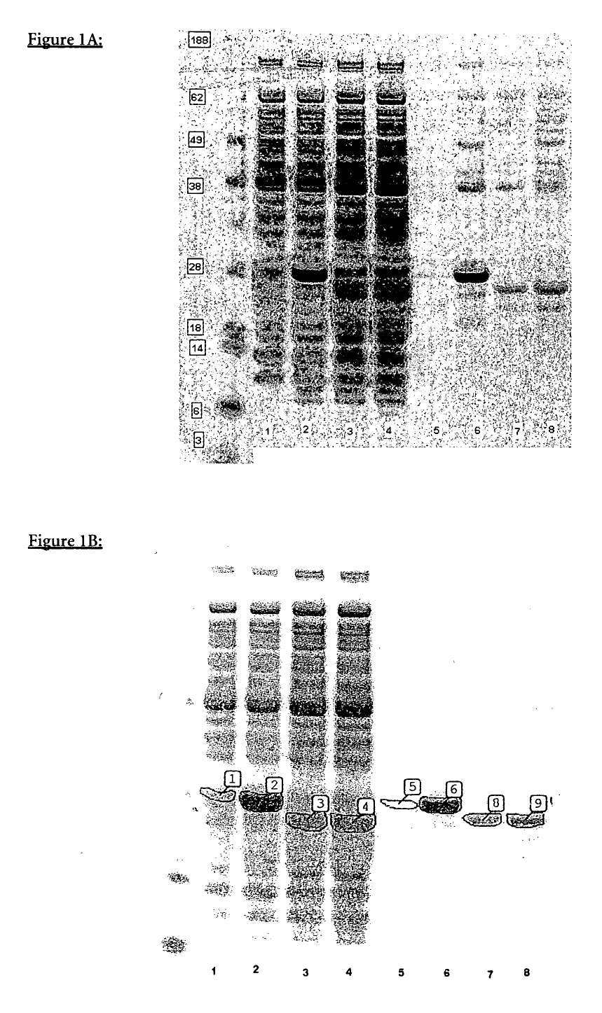

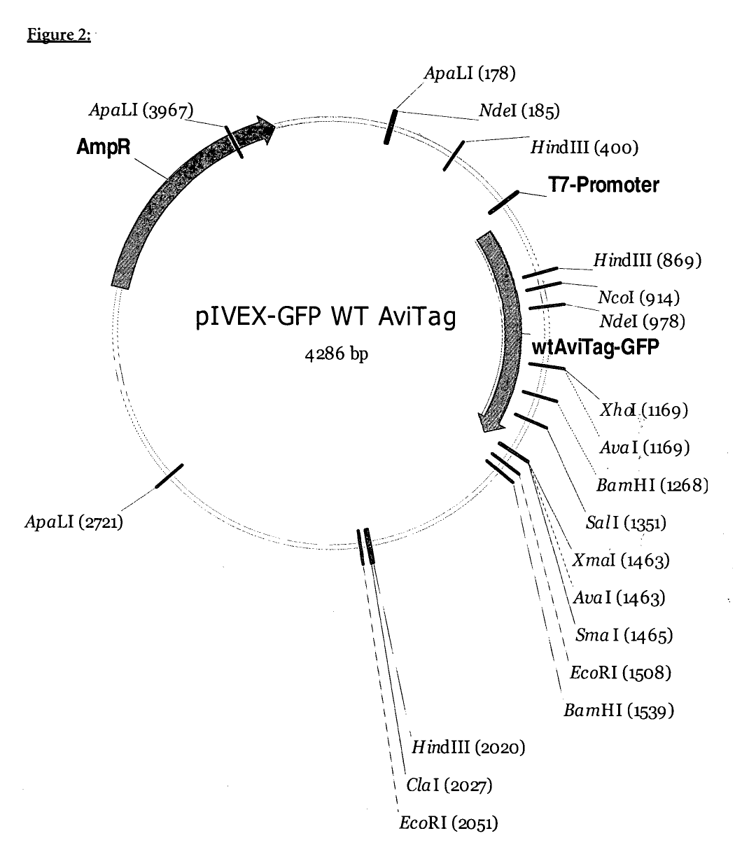

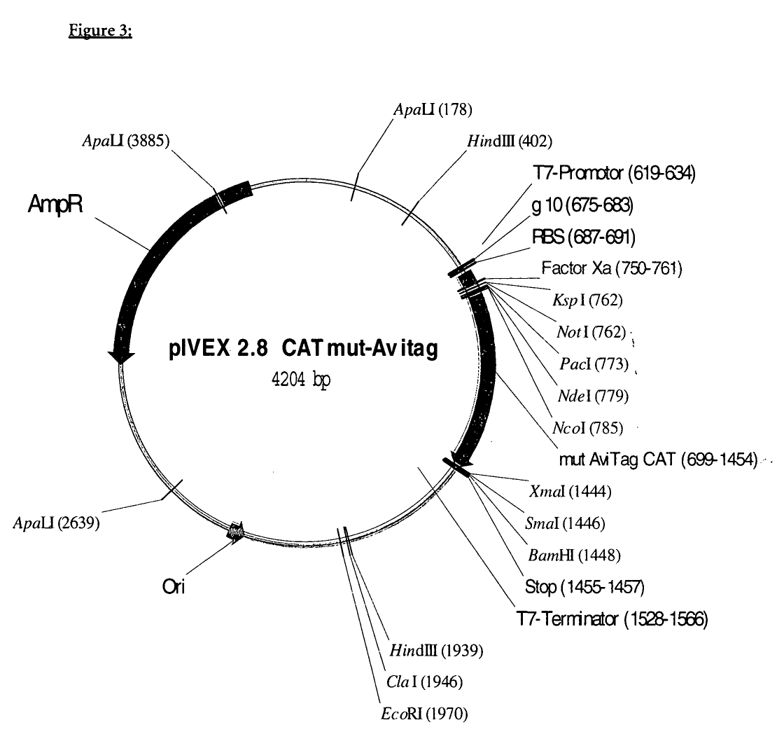

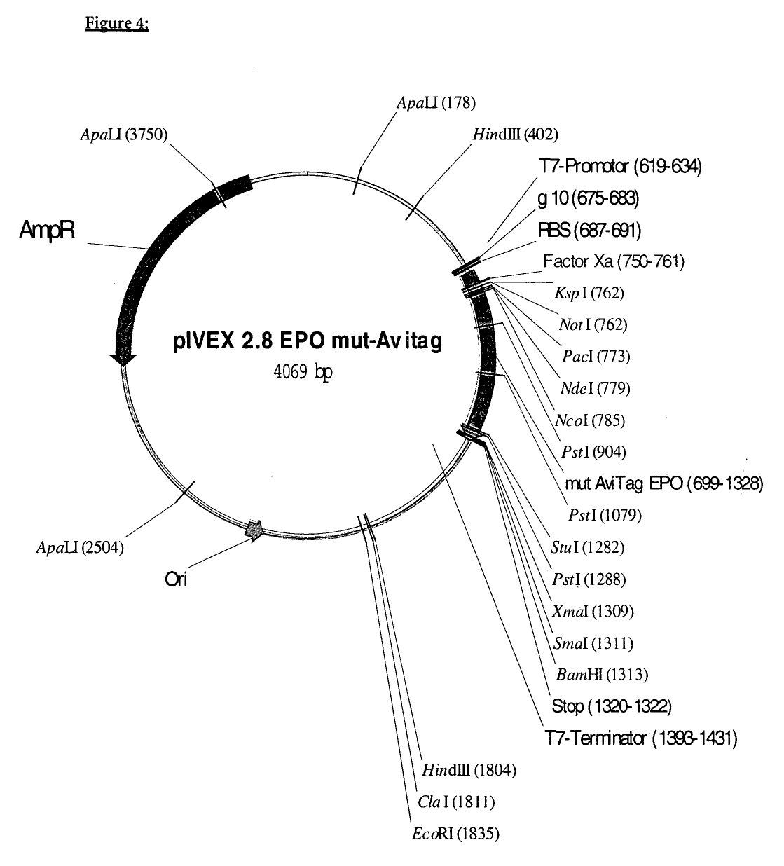

After the run SDS gels were stained with Coomassie Brilliant Blue. FIG. 1 shows the result. The fusion polypeptides encoded by the wildtype AviTag DNA sequence that was operably linked to the coding sequences of either CAT or EPO were present in smaller quantities as opposed to those fusion polypeptides in which the N-terminal tag was encoded by the mutated sequence of SEQ ID NO: 12. EPO in its unglycosylated form can be detected primarily in the pellet fraction. This result exemplifies, that a mutant variant of the DNA sequence encoding the AviTag biotinylation polypeptide, as compared to the wildtype sequence, enhances the formation of a fusion polypeptide, consisting of an N-terminal polypeptide according to SEQ ID NO: 1 and a C-terminal polypeptide with a biological function, by means of expression from a nucleic acid encoding said fusion polypeptide in a cell-free polypeptide synthesis reaction mixture.

### EXAMPLE 4

### Quantification of Expressed Fusion Polypeptides

The amounts of expressed fusion polypeptides were quantified by way of densitometric measurements of coomassie-stained bands in SDS gels that were obtained using the Lumi Imager F1 and the LumiAnalyst Software (Roche Diagnostics GmbH, Mannheim, Germany). Measurements were made according to the instructions of the manufacturer. Each analysed each gel contained control lanes in which defined amounts of marker proteins were electrophoresed in order to provide reference points for quantification. Table 1 provides results from the parallel experiments described in Example 3 and FIG. 1.

TABLE 1
Quantification of fusion polypeptides expressed by
the RTS 500 HY Kit using the expression vectors

| | SDS gel | Densitometric | Concentration |
|---|---|---|---|
| Vector | lane | readout | [mg/ml] |
| pIVEX-2.8 CAT WT AviTag | 1 | 31.841 | 0.5 |
| pIVEX-2.8 CAT mut AviTag | 2 | 237.345 | 6.5 |
| pIVEX-2.8 EPO WT AviTag | 3 | 94.040 | 2.3 |
| pIVEX-2.8 EPO mut AviTag | 4 | 129.975 | 3.3 |
| pIVEX-2.8 CAT WT AviTag | 5 | 5.255 | 0 |
| pIVEX-2.8 CAT mut AviTag | 6 | 188.364 | 5.0 |
| pIVEX-2.8 EPO WT AviTag | 7 | 43.288 | 0.8 |
| pIVEX-2.8 EPO mut AviTag | 8 | 70.833 | 1.6 |

The results indicate that the mutant variant of the wildtype sequence as given in SEQ ID NO: 12 enhances the formation of the fusion polypeptide, consisting of an N-terminal polypeptide according to SEQ ID NO: 1 and a C-terminal polypeptide with a biological function in that at least 40% more fusion polypeptide is formed.

What is claimed is:

1. A nucleic acid sequence comprising a biotinylation sequence, said biotinylation sequence consisting of ATGWSYGGHY TRAAYGAYAT YTTYGAGGCW CAGAAAATCG AATGGCACGAA (SEQ ID NO: 2), wherein W is A or T, S is G or C, Y is T or C, H is A, C or T, and R is G or A, with the proviso that the biotinylation sequence is not SEQ ID NO: 3.

2. The nucleic acid sequence of claim 1 wherein the biotinylation sequence is selected from the group consisting of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID
NO: 22, SEQ ID NO: 23, and SEQ ID NO: 24.

3. An expression vector comprising a promoter operably linked to a biotinylation sequence, said biotinylation sequence consisting of ATGWSYGGHY TRAAYGAYAT YTTYGAGGCW CAGAAAATCG AATGGCACGAA (SEQ ID NO: 2), wherein W is A or T, S is G or C, Y is T or C, H is A, C or T, and R is G or A, with the proviso that the biotinylation sequence is not SEQ ID NO: 3.

4. The expression vector of claim 3 further comprising a synthetic oligonucleotide linker, comprising a plurality of endonuclease restriction sites, operably linked to the 3′ end of SEQ ID NO: 2.

5. The expression vector of claim 3 wherein the promoter is a T7 promoter.

6. The expression vector of claim 3 wherein the biotinylation sequence is selected from the group consisting of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, and SEQ ID NO: 24.

7. The expression vector of claim 6 wherein the biotinylation sequence consists of SEQ ID NO: 12.

8. A method of synthesizing a fusion polypeptide capable of being biotinylated by holocarboxylase synthetase, said method comprising the steps of:
(a) operably linking a first nucleic acid sequence to a second nucleic acid sequence to form a linked sequence, wherein said first nucleic acid sequence comprises a promoter operably linked to a biotinylation sequence, said biotinylation sequence consisting of ATGWSYGGHY TRAAYGAYAT YTTYGAGGCW CAGAAAATCG AATGGCACGAA (SEQ ID NO: 2), wherein W is A or T, S is G or C, Y is T or C, H is A, C or T, and R is G or A, with the proviso that the biotinylation sequence is not SEQ ID NO: 3, and said second nucleic acid sequence encoding a polypeptide; and
(b) expressing said linked sequence to produce said fusion polypeptide.

9. The method of claim 8 wherein said promoter is a T7 promoter.

10. The method of claim 8 wherein the biotinylation sequence is selected from the group consisting of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, and SEQ ID NO: 24.

11. The method of claim 10 wherein the biotinylation sequence consists of SEQ ID NO: 12.

12. The method of claim 8 wherein the second nucleic acid sequence encodes a polypeptide with a biological function.

13. The method of claim 8 wherein the expression takes place within a cell.

14. The method of claim 13 wherein said cell expresses holocarboxylase synthetase.

15. The method of claim 13 wherein said cell is E. coli.

16. The method of claim 8 wherein the expression takes place in vitro in a cell free reaction mixture.

17. A method of preparing a biotinylated polypeptide, said method comprising the steps of:
(a) operably linking a first nucleic acid sequence to a second nucleic acid sequence to form a linked sequence, wherein said first nucleic acid sequence comprises a promoter operably linked to a biotinylation sequence, said biotinylation sequence consisting of ATGWSYGGHY TRAAYGAYAT YTTYGAGGCW CAGAAAATCG AATGGCACGAA (SEQ ID NO: 2), where in W is A or T, S is G or C, Y is T or C, H is A, C or T, R is G or A, with the proviso that the biotinylation sequence is not SEQ ID NO: 3, and said second nucleic acid sequence encoding a polypeptide;
(b) expressing said linked sequence to produce a fusion polypeptide; and
(c) contacting said fusion polypeptide with biotin and holocarboxylase synthetase.

18. The method of claim 17 wherein the expression takes place in vitro in a cell free reaction mixture.

19. The method of claim 18 wherein the holocarboxylase synthetase is supplied as a purified protein.

20. The method of claim 18 wherein a nucleic acid expression vector encoding holocarboxylase synthetase is added to the reaction mixture and holocarboxylase synthetase is co-expressed with the fusion polypeptide.

21. The method of claim 17 further comprising the step of purifying the synthesized fusion polypeptide.
